(19) United States
(12) Patent Application Publication
(10) Pub. No.: US 2005/0187507 A1
(43) Pub. Date: Aug. 25, 2005

(54) Method and apparatus for removal of menses, reduction of menses interference during coitus, and enhancement of sexual pleasure

(76) Inventors: Andre' L. Reed, Alton, IL (US); Sandra Black, Edwardsville, IL (US)

Correspondence Address: Clyde L. Smith;Thompson Coburn LLP, One US Bank Plaza, St. Louis, MO, 63101, US

(21) Appl. No.: 11/030,604

(22) Filed: Jan. 6, 2005

Related U.S. Application Data
(60) Provisional application No. 60/535,193, filed on Jan. 8, 2004.

(51) Int. Cl. A61M 35/00; A61F 5/00; A61F 13/15; A61F 13/20

(52) U.S. Cl. 604/1; 600/38; 604/2; 604/385.17

(57) ABSTRACT

A vaginal cleaning device for the removing menses from a vaginal canal is disclosed. An internal absorbent member configured and adapted to be positioned within the vaginal canal during coitus so as to limit the presence of menses in the vaginal canal during coitus is also disclosed. Still further a cleansing fluid is disclosed for use with the vaginal cleaning device and the internal absorbent member.

This application claims priority from U.S. provisional patent application Ser. No. 60/535,193 filed Jan. 8, 2004 entitled M-pak (incorporated herein in its entirety by reference).

### BACKGROUND OF THE INVENTION

(1) Field of the Invention

This invention pertain to the field of feminine hygiene products. More particularly, this invention pertains to a cleaning device for removing menses from a vaginal canal, and to an absorbent member configured and adapted to be positioned within the vaginal canal during vaginal intercourse so as to limit the presence of menses in the vaginal canal during such intercourse and to mechanically stimulate vaginal mucosa during intercourse.

(2) General Information

It is not uncommon for women to experience a heighten sense of sexual arousal during menstruation. Additionally, such women may also experience greater sexual pleasure in response to sexual stimulation during menstruation. This is due, at least in part, to hormonal surges that coincide with menstruation cycles.

However, many women and their partners avoid coitus and other sexual activities during menstruation because of psychological discomfort attributed to menses. In particular, women and men often find the menses embarrassing. Additionally, the appearance, scent, texture, and/or taste of menses can be repugnant to both men and women.

In view of the foregoing, various products have been developed to facilitate the removal of menses from vaginal cavities. Additionally, other products have been developed to prevent or limit the draining of menses from vaginal cavities. However, such products often make coitus impossible or do not eliminate the possibility of additional menses draining during sexual activities. Moreover, such products fail to eliminate all of the inhibitions sexual partners often have regarding engaging in sexual activities during menstruation. Thus, there remains a need for new and better devices and methods for removing menses from vaginal cavities and for preventing menses draining during sexual activities.

### SUMMARY OF THE INVENTION

The devices and methods of the present invention provide means for overcoming the problems associated with coitus and other sexual activities during menstruation and the disadvantage of prior art products. In general, a unique vaginal cleaning device is disclosed for removing menses from vaginal cavities. Additionally, a unique absorbent member is disclosed that can be positioned within a vaginal cavity in a manner allowing coitus while also preventing or limiting menses drainage. Furthermore, the absorbent member is preferably impregnated with a composition that enhances sexual arousal. Still further, the absorbent member enhances mechanical stimulation of vaginal mucosa during coitus.

In a first aspect of the invention, a method comprises the steps of providing a vaginal cleaning device and promoting the vaginal cleaning device being useful for removing menses from human vaginas. The vaginal cleaning device comprises a wand portion and an absorbent portion. The wand portion of the vaginal cleaning device comprises a handle portion and an elongate portion. The handle portion is adapted and configured to be grasped by a person's hand. The elongate portion extends from the handle portion and has a terminal end portion that is spaced from the handle portion. The absorbent portion is secured to the terminal end portion of the elongate portion and is adapted and configured to absorb fluid. The absorbent portion and at least part of the elongate portion of the wand portion are configured and adapted to be inserted within a human vagina. The elongate portion is sufficiently rigid such that the absorbent portion can be articulated within the vagina by applying a force to the handle portion of the wand.

In another aspect of the invention, a method comprises the steps of providing an absorbent member and promoting the absorbent member as being useful for engaging in sexual activities during menstruation. The absorbent member is adapted and configured to absorb fluid and to be inserted completely within a human vagina in a manner such that coitus can occur while the absorbent member is inserted completely within the vagina.

In yet another aspect of the invention, an absorbent member is adapted and configured to absorb fluid and to be inserted completely within a human vagina in a manner such that coitus can occur while the absorbent member is inserted completely therein. The absorbent member comprises an absorbent portion and a retrieval portion. The absorbent member also comprises a loop that is at least partially defined by the retrieval portion. The loop forms an opening that is adapted and configured to allow a human finger to be inserted therethrough and to pull on the loop.

While the principal advantages and features of the invention have been described above, a more complete and thorough understanding of the invention may be obtained by referring to the drawings and the detailed description of the preferred embodiment, which follow.

### BRIEF DESCRIPTION OF THE DRAWINGS

FIG. 1 is perspective view of a vaginal cleaning device in accordance with the invention.

FIG. 2 is a cross-sectional view of the vaginal cleaning device shown in FIG. 1.

FIG. 3 is a perspective view of an internal absorbent member in accordance with the invention.

FIG. 4 is a perspective view showing the construction of the internal absorbent member shown in FIG. 3.

Reference characters in the written specification indicate corresponding items shown throughout the drawing figures.

### DETAILED DESCRIPTION OF THE PREFERRED EMBODIMENT OF THE INVENTION

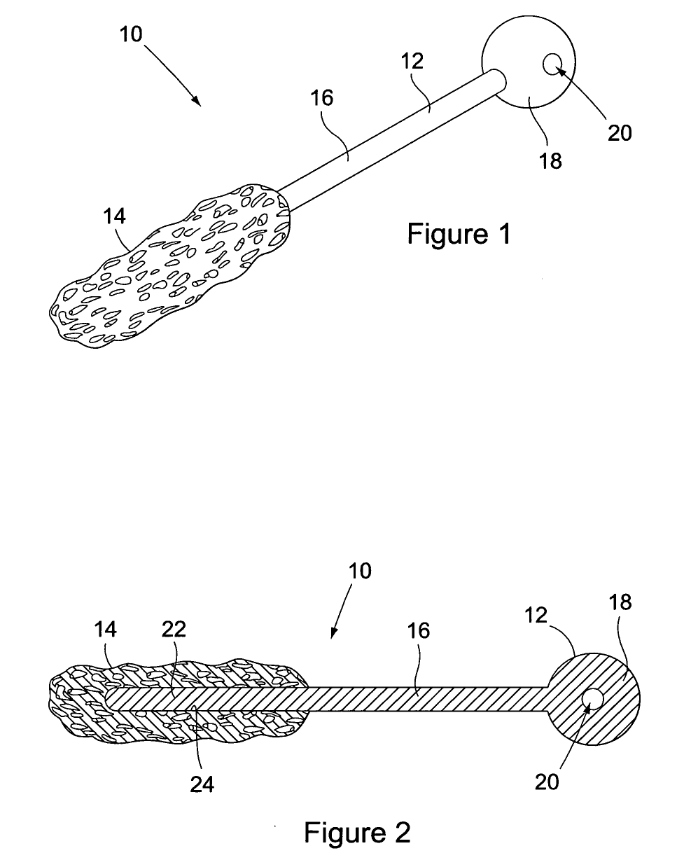

A vaginal cleaning device 10 in accordance with the invention is shown in FIGS. 1 and 2. In general, the vaginal cleaning device 10 preferably comprises a wand portion 12 and an absorbent portion 14.

The wand portion 12 of the vaginal cleaning device 10 is preferably formed as a monolithic piece of polymeric resin, such as polyethylene or nylon via injection molding. The wand portion 12 preferably comprises a elongate portion 16 and a handle portion 18. The handle portion 18 is preferably spherical and preferably comprises an opening 20 that extends therethrough. The opening 20 allows a lanyard or the like to be attached to the handle portion 18 or can be utilized to attach the device 10 to other articles. The elongate portion 16 preferably has a circular cross-section and extends linearly from the handle portion 18. The elongate portion 16 preferably has a terminal end portion 22 opposite the handle portion 18 that is rounded or chamfered such that it is devoid of sharp edges.

The absorbent portion 14 of the vaginal cleaning device 10 is preferably generally cylindrical in shape with rounded ends and is preferably formed of natural sponge material. A blind hole 24 is preferably formed axially into the absorbent portion 14. The terminal end portion 22 of the elongate portion 16 of the wand 12 is positioned in the blind hole 24 absorbent portion and is adhered to the absorbent portion 14 via a suitable non-toxic adhesive.

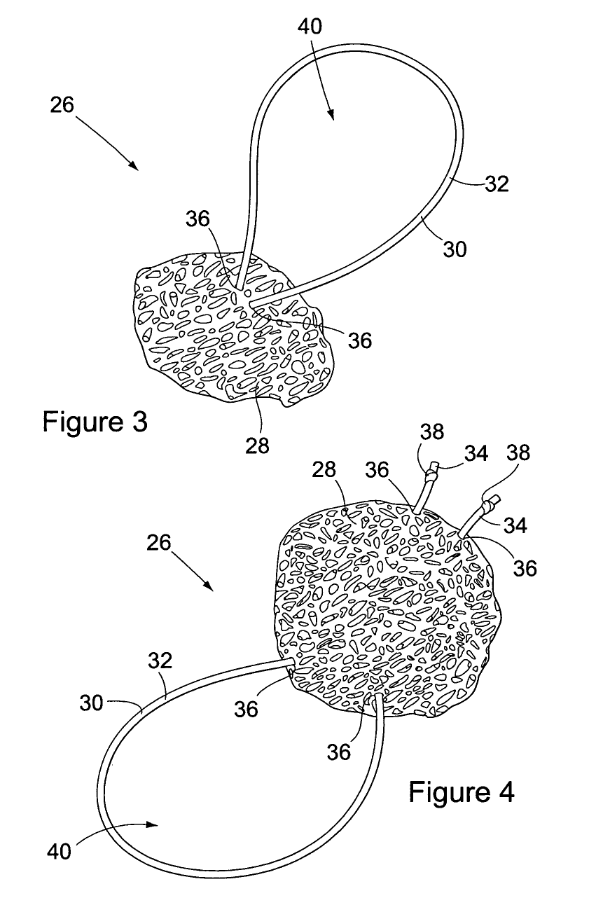

An internal absorbent member 26 in accordance with the invention is shown in FIGS. 3 and 4. The internal absorbent member 26 preferably comprises an absorbent portion 28 and a retrieval portion 30. The absorbent portion 28 of the absorbent member 26 is preferably formed out of a piece of natural sponge material and is preferably generally spherical in shape. The retrieval portion 30 is preferably a strap of flexible material 32 having opposite ends 34. The strap of flexible material 32 is preferably formed of one or more fibers of nontoxic polymeric material and may have any cross-sectional shape. As shown in FIG. 4, the absorbent member is preferably assembled by passing the ends 34 of the strap of flexible material 32 through openings 36 that extend through the absorbent portion 28, and thereafter forming a knot 38 adjacent to each of the ends. It should be appreciated that the openings 38 may be openings that were formed as a natural part of the sponge. Alternatively, the openings 38 may be formed by removing material from the absorbent portion 28 or may otherwise be formed during the creation of the absorbent portion. The ends 34 of the strap of flexible material 32 are then pulled partially back through the absorbent portion 28 such that the knots 38 are surrounded by the absorbent portion 28. It should be appreciated that the cross-section dimension of the openings 36 through which the ends 34 of the strap of flexible material 32 are inserted is preferably less that the diameter of the knots 38 such that an appreciable force is required to pass the knots through the openings. This acts to secure the strap of flexible material 32 to the absorbent portion 28. If needed or desired, the ends 34 of the strap of flexible material 32 can be further secured to the absorbent portion 28 by passing stitches through the absorbent portion and the strap of flexible material 32 using non-toxic thread. Once assembled, the strap of flexible material 32 is secured to the absorbent portion 28 and forms a loop that defines and encircles an opening 40 exterior to the absorbent portion.

In accordance with the invention, the absorbent portion 28 of the absorbent member 26 is preferably impregnated with a sexual enhancement composition (not shown). The sexual enhancement composition can be originally applied to the absorbent portion 28 in fluid form and thereafter dried, cured, or left in a fluid state. Alternatively, the sexual enhancement composition can be encapsulated in a non-toxic breakable or dissolvable capsule that is inserted in a hole or opening within the absorbent portion. The sexual enhancement composition preferably comprises an assortment of vitamins, lubricants, gustatory an other stimulants, and aromatic substances. More particularly, the sexual enhancement composition preferably comprises the following ingredients:
L-Arginine (vasodilator) 3.5 g
Avena Sativa (aphrodisiac/strength enhancer) 100 mg
Urtica Dioica (circulatory stimulant) 100-300 mg
Yohimbe (aphrodisiac/strength enhancer) 100-300 mg
Aspidosperma Quebracho (respiratory enhancer) 100-300 mg
Siberian Ginseng (antianxiety/strength enhancer) 50-100 mg
Fructose (energy) 10 mg
Glucose 6 Phosphate Dehydrogenase (enzyme supplement) 50 mg
Peppermint/Spearmint/Tea Tree Oil (flavoring/menthol source)
Menthol (mild topical anesthetic) 10 mg
Black Licorice (flavoring) 50-100 mg
Goat weed Extract (cell stimulant) 100-300 mg
Petroleum Jelly/mineral oil (lubricant/carrier)
A-Angelica Lactone 50-100 mg
Tocopheryl Nicotinate 100-300 mg
Niacin 25-200 mg
Coenzyme Q10 (energizer) 100-300 mg
Ginkgo Biloba Extract 10-50 mg 50:1
Procaine 30-50 mg
Sodium Bicarbonate

Having described embodiments of a vaginal cleaning device 10, an internal absorbent member 26, and a sexual enhancement composition in accordance with the invention, a description of the method of using such items herein follows. The primary utility of the vaginal cleaning device 10, internal absorbent member 26, and sexual enhancement composition is to reduce or eliminate the unpleasantness associated with menses flow for the purpose of facilitating sexual activity, including coitus, during menstruation. Additionally, the internal absorbent member 26 and the sexual enhancement composition are adapted and configure to enhance and stimulate sexual pleasure.

Preferably, a woman desiring to engage in coitus or other sexual activities during menstruation first moistens the absorbent portion 14 of the vaginal cleaning device 10 with water or other non-toxic liquids while holding the vaginal cleaning device by the handle 18 of its wand portion 12. The absorbent portion 14 of the vaginal cleaning device 10 releasably retains such liquids.

After having moistened the absorbent portion 14 of the vaginal cleaning device 10, the woman then inserts the absorbent portion into her vaginal cavity by applying a force on the handle portion 18 of the vaginal cleaning device. The liquid used to moisten absorbent portion 14 of the vaginal cleaning device 10 facilitates such insertion by reducing frictional forces. Once the absorbent portion 14 is positioned within the vaginal cavity, the woman is then able to move the absorbent portion in a reciprocating manner via the handle 18 of the wand 12 so that the absorbent portion lightly rubs against at least a majority of the vaginal wall. The cylindrical diameter of the absorbent portion 14 is preferably dimensioned in a manner such that the vaginal wall engages around the absorbent portion and thereby partially compresses the absorbent portion during this step. This facilitates both the expulsion and absorption of fluid from the absorbent portion 14 during this step and creates a pressure force on the vaginal wall. It is envisioned that multiple variations of vaginal cleaning devices having differently dimensioned absorbent portions may be made available for this purpose. It should also be appreciated that the elongate portion 16 of the wand 12 of the vaginal cleaning device 10 has a length sufficient to allow the absorbent portion 14 to engage all portions of the vaginal wall while the handle 18 of the wand remains outside of the vaginal cavity. This step helps remove any menses, including clots, from the vaginal wall by transferring such menses to the absorbent portion 14 of the vaginal cleaning device 10.

Following the above described steps, the vaginal cleaning device 10 is removed from the vaginal cavity. If desired, those steps can be repeated using a new vaginal cleaning device or by reusing the vaginal cleaning device 10 after rinsing or otherwise cleaning the absorbent portion 14 of the vaginal cleaning device. Thereafter, the woman preferably moistens the absorbent portion 28 of the internal absorbent member 26 via water or other non-toxic liquids in manner similar to the moistening of the absorbent portion 14 of the vaginal cleaning device 10. The moistening of the absorbent portion 28 of the internal absorbent member 26 acts to activate the sexual enhancement composition. If the sexual enhancement composition is provided within a capsule, the moistening of the absorbent portion 28 may dissolve the capsule, or the capsule may be ruptured by squeezing to thereby release the sexual enhancement composition. Furthermore, it is envisioned that internal absorbent members 26 may be individually packaged in moisture proof packaging and be pre-moistened therewithin.

With the internal absorbent member 26 moistened and the sexual enhancement composition activated, the internal absorbent member is inserted completely into the vaginal cavity. Preferably, the lubricating properties of at least some of the ingredients of the sexual enhancement composition facilitate such insertion by reducing frictional forces. The internal absorbent member 26 is preferably positioned sufficiently deep within the vaginal cavity to permit coitus while positioned therein. The woman preferably does this by applying a pushing force on the internal absorbent member 26. The absorbent portion 28 of the internal absorbent member 26 is preferably dimensioned in a manner such that it remains slightly radially compressed by the vaginal wall and thereby forms a barrier to menses flow therearound. Thus, it should also be appreciated that, as the internal absorbent member 26 is pushed along the vaginal cavity, any residual menses along the portion of the vaginal wall that the internal absorbent member passes is forced deeper into the vaginal cavity or is absorbed by the absorbent portion of the internal absorbent member. Additionally, the absorbent portion 28 of the internal absorbent member 26 is also preferably configured and adapted to absorb approximately 40 ml of fluid. Thus, it should be appreciated that the internal absorbent member 26 thereafter prevents or limits any additional menses from flowing into the lower vaginal cavity and can absorb an appreciable amount of menses.

Having performed the steps discussed above, the woman is able to engage in coitus and other sexual activities without the worries or uneasiness normally associated with conducting such activities during menstruation. Moreover, the sexual enhancement composition described above lubricates the vaginal cavity and acts as a stimulant, thereby further enhancing sexual pleasure. Furthermore, the sexual enhancement composition has an aroma and taste that can further increase sexual arousal and pleasure. Still further, during coitus, the penis can contact the internal absorbent member 26 and thereby stimulate the portion of the vaginal wall that is in contact with the absorbent member. This increases the overall surface area of the vaginal wall that can be stimulated during coitus. Thus, it should be appreciated that it may be desirable to utilize the internal absorbent member 26 when engaging in sexual activities regardless of whether the timing of such activities coincide with menstruation.

After use, the internal absorbent member 26 can easily be removed from the vaginal cavity utilizing the retrieval portion 30. This is performed by inserting a finger into the vaginal cavity and applying a pulling force on the strap of flexible material 32 by hooking the finger through the opening 40 created thereby. After removing the internal absorbent member 26 from the vaginal cavity, the vaginal cleaning device 10 can be utilized as described above to remove any undesired residual menses.

The foregoing methods of using a vaginal cleaning device 10, an internal absorbent member 26, and sexual enhancement composition in accordance with the invention is preferably promoted in or on the packaging of such products via text, indicia, or other well known means of communicating. Additionally, it is envisioned that these products can be sold individually or together and can be combined with other products as desired.

While the present invention has been described in reference to specific embodiments, in light of the foregoing, it should be understood that all matter contained in the above description or shown in the accompanying drawings is intended to be interpreted as illustrative and not in a limiting sense and that various modifications and variations of the invention may be constructed without departing from the scope of the invention defined by the following claims. Thus, other possible variations and modifications should be appreciated.

Furthermore, it should be understood that when introducing elements of the present invention in the claims or in the above description of the preferred embodiment of the invention, the terms “comprising,” “including,” and “having” are intended to be open-ended and mean that there may be additional elements other than the listed elements. Similarly, the term “portion” should be construed as meaning some or all of the item or element that it qualifies.

1. A method comprising:
providing a vaginal cleaning device, the vaginal cleaning device comprising a wand portion and an absorbent portion, the wand portion comprising a handle portion and an elongate portion, the handle portion being adapted and configured to be grasped by a person's hand, the elongate portion extending from the handle portion and having a terminal end portion that is spaced from the handle portion, the absorbent portion being secured to the terminal end portion of the elongate portion and being adapted and configured to absorb fluid, the absorbent portion and at least part of the elongate portion of the wand portion being configured and adapted to be inserted within a human vagina, the elongate portion being sufficiently rigid such that the absorbent portion can be articulated within the vagina by applying a force to the handle portion of the wand portion; and
promoting the vaginal cleaning device being useful for removing menses from human vaginas.

2. A method in accordance with claim 1 wherein the step of providing the vaginal cleaning device occurs in a manner such that the absorbent portion comprises an opening that extends thereinto and at least partially therethrough, and in a manner such that the terminal end portion of the elongate portion is positioned within the opening of the absorbent portion.

3. A method in accordance with claim 1 wherein the step of providing the vaginal cleaning device occurs in a manner such that the absorbent portion comprises natural sponge material.

4. A method in accordance with claim 1 further comprising a step of providing packaging for the vaginal cleaning device, and wherein the step of promoting the vaginal cleaning device as being useful for removing menses from human vaginas comprises providing indicia on the packaging that promotes the vaginal cleaning device being useful for removing menses.

5. A method in accordance with claim 1 further comprising a step of providing an absorbent member, the absorbent member being adapted and configured to absorb fluid and to be inserted completely within a human vagina in a manner such that coitus can occur while the absorbent member is inserted completely within the vagina, and steps of packaging the vaginal cleaning device together with the absorbent member and promoting the absorbent member as being useful for engaging in sexual activities during menstruation.

6. A method in accordance with claim 5 wherein the step of providing the absorbent member comprises impregnating at least a portion of the absorbent member with a sexual enhancement composition.

7. A method in accordance with claim 6 wherein the step of promoting the absorbent member as being useful for engaging in sexual activities during menstruation comprises promoting the sexual enhancement composition as being capable of enhancing sexual arousal.

8. A method comprising:
providing an absorbent member, the absorbent member being adapted and configured to absorb fluid and to be inserted completely within a human vagina in a manner such that coitus can occur while the absorbent member is inserted completely within the vagina; and
promoting the absorbent member as being useful for engaging in sexual activities during menstruation.

9. A method in accordance with claim 8 wherein the step of providing the absorbent member occurs in a manner such that the absorbent member comprises an absorbent portion and a retrieval portion, the retrieval portion forming at leat a partially closed loop and being secured to the absorbent portion in manner such that the absorbent member can be removed from a vagina by exerting a pulling force on the loop.

10. A method in accordance with claim 9 wherein the step of providing the absorbent member occurs in a manner such that the absorbent potion comprises a pair of openings that extend through the absorbent portion, and such that the loop of the retrieval portion extends through the pair of openings in a manner interlocking the retrieval portion to the absorbent portion.

11. A method in accordance with claim 8 wherein the step of providing the absorbent member occurs in a manner such that the absorbent portion comprises natural sponge material.

12. A method in accordance with claim 8 further comprising a step of providing packaging for the absorbent member, and wherein the step of promoting the absorbent member as being useful for engaging in sexual intercourse during menstruation comprises providing indicia on the packaging that promotes the absorbent member as being useful for engaging in sexual activities during menstruation.

13. A method in accordance with claim 8 wherein the step of providing the absorbent member comprises impregnating at least a portion of the absorbent member with a sexual enhancement composition, and wherein the step of promoting the absorbent member as being useful for engaging in sexual activities during menstruation comprises promoting the sexual enhancement composition as being capable of enhancing sexual arousal.

14. A method in accordance with claim 8 wherein the step of promoting the absorbent member as being useful for engaging in sexual activities during menstruation comprises promoting the absorbent member as being capable of mechanically stimulating vaginal mucosa during coitus.

15. A method in accordance with claim 8 further comprising a step of providing a vaginal cleaning device, the vaginal cleaning device comprising a wand portion and an absorbent portion, the wand portion comprising a handle portion and an elongate portion, the handle portion being adapted and configured to be grasped by a person's hand, the elongate portion extending from the handle portion and having a terminal end portion that is spaced from the handle portion, the absorbent portion being secured to the terminal end portion of the elongate portion and being adapted and configured to absorb fluid, the absorbent portion and at least part of the elongate portion of the wand portion being configured and adapted to be inserted within a human vagina, and steps of packaging the vaginal cleaning device together with the absorbent member and promoting the vaginal cleaning device being useful for removing menses from human vaginas.

16. An absorbent member, the absorbent member being adapted and configured to absorb fluids and to be inserted completely within a human vagina in a manner such that coitus can occur while the absorbent member is inserted completely within the vagina, the absorbent member comprising an absorbent portion and a retrieval portion, the absorbent member comprising also comprising a loop that is at least partially defined by the retrieval portion, the loop forms an opening that is adapted and configured to allow a human finger to be inserted therethrough and to pull on the loop.

17. An absorbent member in accordance with claim 16 wherein the absorbent portion of the absorbent member is a monolithic piece of sponge material, the piece of sponge material having an exterior surface and an internal volume, the piece of sponge material also has a pair of spaced apart openings that extend therethrough, the retrieval portion comprises a strap member that extends through each of the pair of spaced apart openings.

18. An absorbent member in accordance with claim 17 wherein the strap has opposite ends and each of the ends forms a knot.

19. An absorbent member in accordance with claim 16 wherein the absorbent portion of the absorbent member is impregnated with a sexual enhancement composition.

20. An absorbent member in accordance with claim 16 wherein the absorbent portion is at least partially comprises natural sponge.

21. A method comprising:
providing an absorbent member, the absorbent member being adapted and configured to absorb fluid and to be inserted completely within a human vagina in a manner such that coitus can occur while the absorbent member is inserted completely within the vagina; and
promoting the absorbent member as being capable of mechanically stimulating vaginal mucosa during coitus.
